(19) United States
(12) Patent Application Publication
(10) Pub. No.: US 2019/0307913 A1
(43) Pub. Date: Oct. 10, 2019

(54) SCENT DIFFUSER USING BOTTLE

(71) Applicant: DONGGUAN YIH TEH ELECTRIC PRODUCTS CO., LTD., Dongguan City (CN)

(72) Inventor: Ming Jen Hsiao, Miaoli County (TW)

(21) Appl. No.: 15/949,688

(22) Filed: Apr. 10, 2018

(51) Int. Cl. A61L 9/12; B65D 83/16; B65D 83/60; F21V 33/00

(52) U.S. Cl. CPC A61L 9/12; B65D 83/16; A61L 2209/11; F21V 33/0024; B65D 2203/00; B65D 83/60

(57) ABSTRACT

A scent diffuser contains: a body, a power connection unit, a pump, and a diffusing device. The body includes an accommodation chamber and a first opening. The power connection unit is housed in the accommodation chamber. The pump includes an air inflow segment and an air outflow segment. The diffusing device is accommodated in the body and includes a cap, a nozzle, a spraying member, a holder, and a spray hose. The cap has a first orifice, a second orifice, and a third orifice. The spraying member has a receiving groove, a suction mouth, and an outlet. The nozzle has a shrinkage conduit and a narrow conduit, and a slit is defined between the nozzle of the diffusing device and the receiving groove of the spraying member. The holder has a connection portion, and the cap is coupled on the holder.

### FIELD OF THE INVENTION

The present invention relates to a scent diffuser, and more particularly to the scent diffuser using a bottle which diffuses scents quickly after spraying and atomizing aromatic liquid by using the diffusing device.

### DESCRIPTION OF THE PRIOR ART

A conventional aroma diffuse is disclosed in U.S. Pat. No. 9,539,355, but its structure is complicated and has a large size. A conventional detachable aromatic nebulizing diffuser is disclosed in U.S. Pat. No. 7,963,460 and contains a fan configured to deliver scents toward an external space, but it consumes power of two electric devices. A lamp-based scent releasing system is disclosed in U.S. Pat. No. 8,201,957 and contains a heating element configured to heat aromatic substance, for example, an electric resistance heats the aromatic substance so as to diffuse scents, thus causing power consumption and heating danger.

The present invention has arisen to mitigate and/or obviate the afore-described disadvantages.

### SUMMARY OF THE INVENTION

The primary aspect of the present invention is to provide a scent diffuser using a bottle which diffuses scents quickly after spraying and atomizing aromatic liquid by using the diffusing device.

To obtain the above aspect, a scent diffuser using a bottle provided by the present invention contains: a body, a power connection unit, a pump, and a diffusing device.

The body includes an accommodation chamber defined in the body and a first opening formed on a top of the accommodation chamber.

The power connection unit is housed in the accommodation chamber of the body.

The pump includes an air inflow segment and an air outflow segment, and the pump is housed in the accommodation chamber of the body and is electrically connected with the power connection unit.

The diffusing device is accommodated in an upper end of the body, and the diffusing device includes a cap, a nozzle, a spraying member, a holder, and a spray hose.

The cap has a first orifice, a second orifice, and a third orifice, the second orifice is formed on a top of the cap and communicates with the outflow segment of the pump, and the third orifice is formed on a bottom of the cap and communicates with the second orifice.

The spraying member has a receiving groove, a suction mouth, and an outlet, wherein the receiving groove is defined in the spraying member, the outlet communicates with a first side of the receiving groove, and the suction mouth is in communication with a second side of the receiving groove.

The nozzle has a shrinkage conduit and a narrow conduit, the shrinkage conduit communicates with the narrow conduit, the spraying member is connected in the holder, the nozzle is accommodated in the receiving groove of the spraying member, a slit is defined between the nozzle and the receiving groove, and a free end of the narrow conduit is proximate to the outlet;

The holder has a connection portion arranged on a side of the holder.

The cap is coupled on a top of the holder, the third orifice of the cap communicates with an upper end of the nozzle, and the first orifice of the cap communicates with an interior of the diffusing device and an exterior outside the diffusing device. A first side of the spray hose communicates with the first orifice, and a second side of the spray hose is in communication with an external environment outside the scent diffuser.

### BRIEF DESCRIPTION OF THE DRAWINGS

FIG. 1 is a perspective view showing the assembly of a scent diffuser using a bottle according to a preferred embodiment of the present invention.

FIG. 2 is a perspective view showing the exploded components of the scent diffuser using the bottle according to the preferred embodiment of the present invention.

FIG. 3 is another perspective view showing the exploded components of the scent diffuser using the bottle according to the preferred embodiment of the present invention.

FIG. 4 is a side plane view of a diffusing device of the scent diffuser using the bottle according to the preferred embodiment of the present invention.

FIG. 5 is a cross sectional view taken along the line A-A of FIG. 4.

FIG. 6 is a side plane view showing the application of the scent diffuser using the bottle according to the preferred embodiment of the present invention.

FIG. 7 is a cross sectional view taken along the line B-B of FIG. 6.

FIG. 8 is a perspective view showing the application of the scent diffuser using the bottle according to the preferred embodiment of the present invention.

### DETAILED DESCRIPTION OF THE PREFERRED EMBODIMENTS

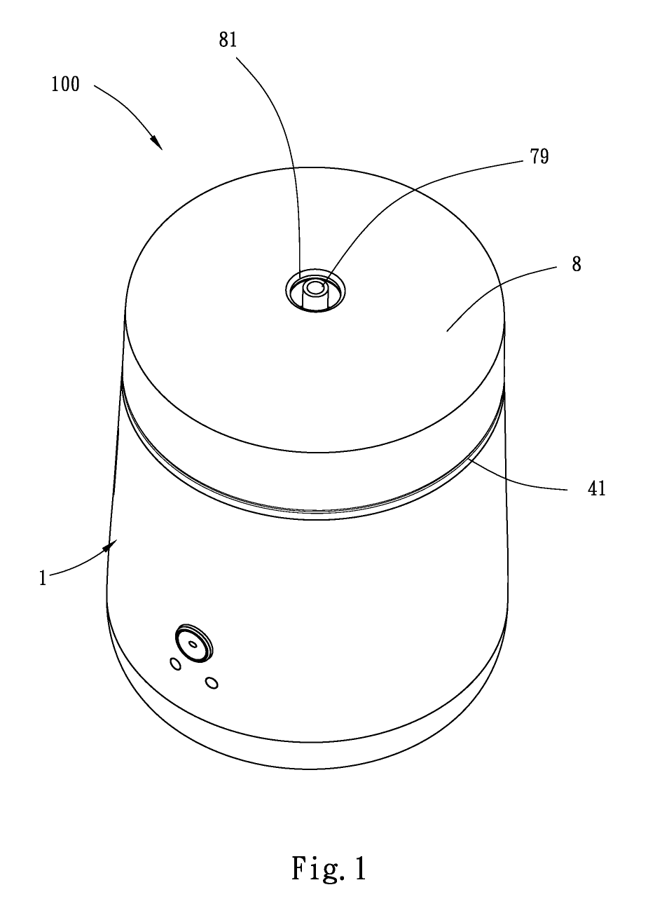

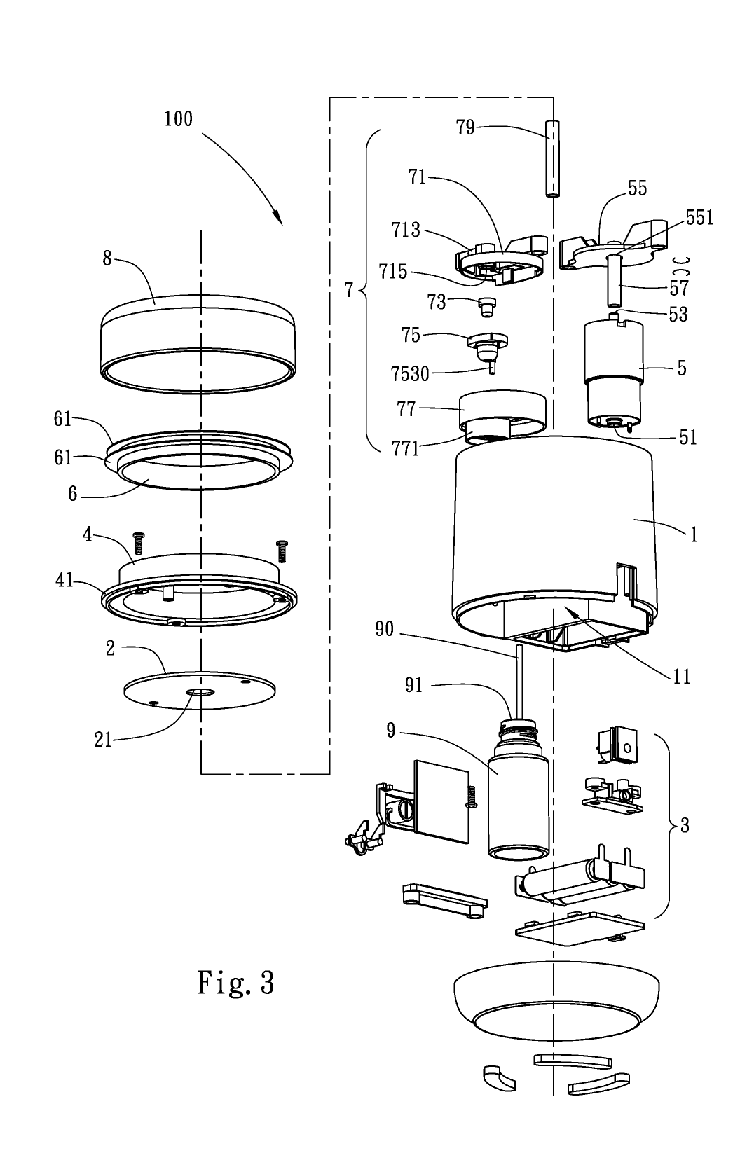

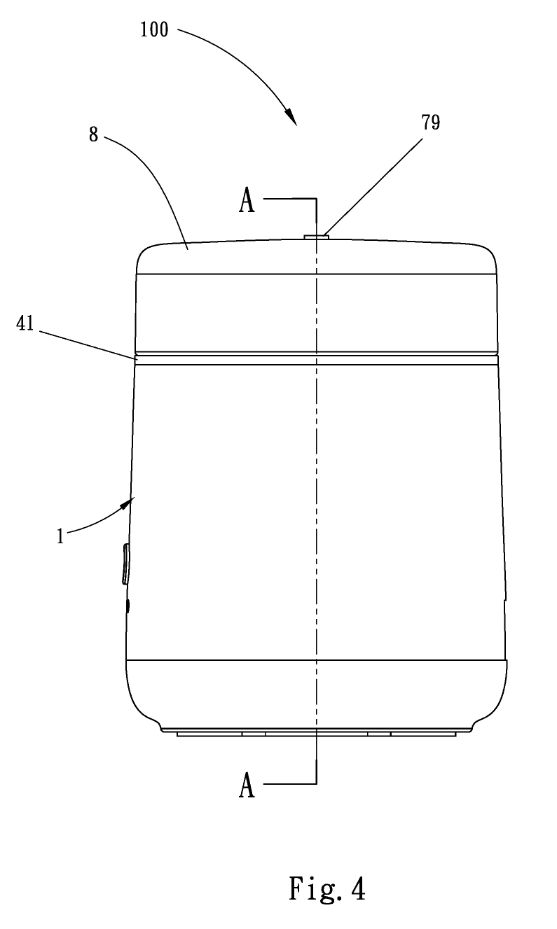

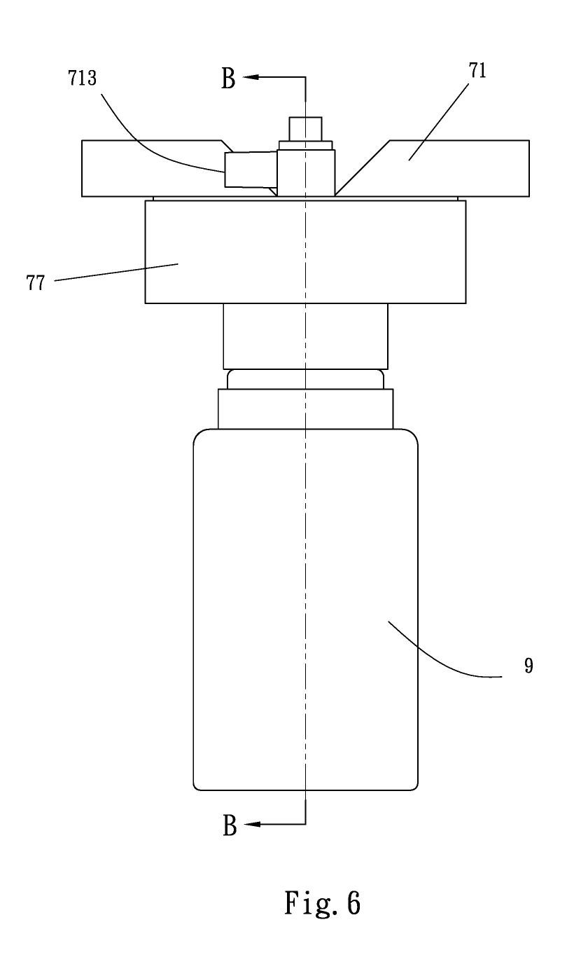

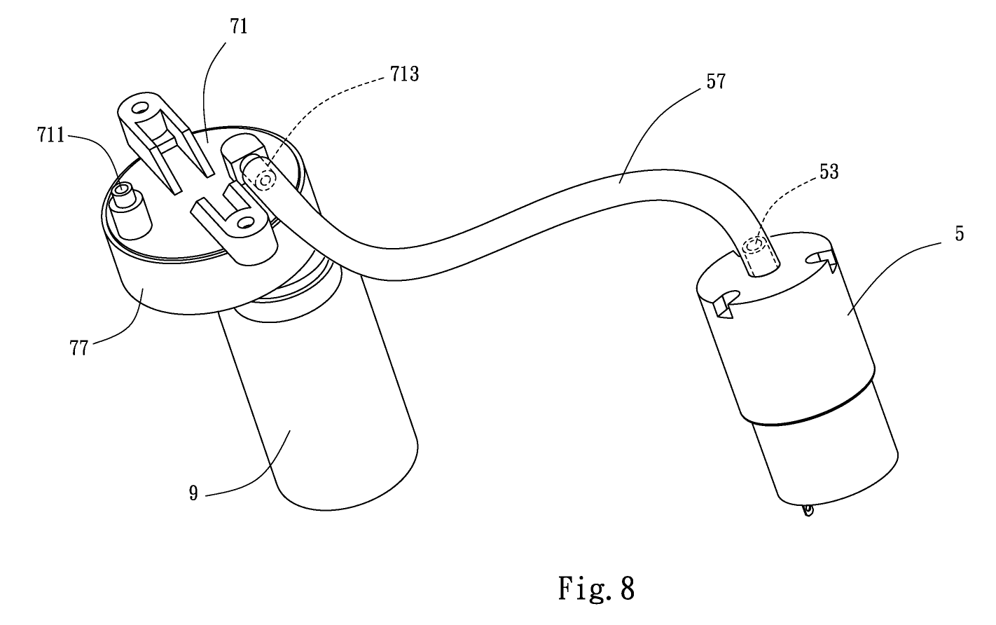

With reference to FIGS. 1-8, a scent diffuser 100 according to a preferred embodiment of the present invention comprises: a body 1 including an accommodation chamber 11 defined in the body 1 and a first opening 13 formed on a top of the accommodation chamber 11;

a power connection unit 3 housed in the accommodation chamber 11 of the body 1;

a pump 5 including an air inflow segment 51 and an air outflow segment 53, and the pump 5 being housed in the accommodation chamber 11 of the body 1 and electrically connected with the power connection unit 3 so as to pump gas successively;

a diffusing device 7 accommodated in an upper end of the body 1, and the diffusing device 7 including a cap 71, a nozzle 73, a spraying member 75, a holder 77, and a spray hose 79;

the cap 71 having a first orifice 711, a second orifice 713, and a third orifice 715, wherein the second orifice 713 is formed on a top of the cap 71 and communicates with the outflow segment 53 of the pump 5, and the third orifice 715 is formed on a bottom of the cap 71 and communicating with the second orifice 713;

the spraying member 75 having a receiving groove 751, a suction mouth 753, and an outlet 755, wherein the receiving groove 751 is defined in the spraying member 75, the outlet 755 communicates with a first side of the receiving groove 751, and the suction mouth 753 is in communication with a second side of the receiving groove 751;

the nozzle 73 having a shrinkage conduit 731 and a narrow conduit 733, wherein the shrinkage conduit 731 communicates with the narrow conduit 733, the spraying member 75 is connected in the holder 77, the nozzle 73 is accommodated in the receiving groove 751 of the spraying member 75, a slit 757 is defined between the nozzle 73 and the receiving groove 751, and a free end of the narrow conduit 733 is proximate to the outlet 755;

the holder 77 having a connection portion 771 arranged on a side of the holder 77;

wherein the cap 71 is coupled on a top of the holder 77, the third orifice 715 of the cap 71 communicates with an upper end of the nozzle 73, and the first orifice 711 of the cap 71 communicates with an interior of the diffusing device 7 and an exterior outside the diffusing device 7; a first side of the spray hose 79 communicates with the first orifice 711, and a second side of the spray hose 79 is in communication with an external environment outside the scent diffuser 100.

A bottle 9 is connected with the diffusing device 7 by way of a sucking tube 90, the bottle 9 includes the sucking tube 90 and a second opening 91 configured to removably connect with the connection portion 771 of the holder 77. The bottle 9 is configured to accommodate aromatic liquid, and the sucking tube 90 is housed in the bottle 9 and a free end of the sucking tube 9 is coupled with the suction mouth 753.

The power connection unit 3 is in connection with a power supply so as to drive a pump 5 to successively pump the gas toward the shrinkage conduit 731 via the second orifice 713 and the third orifice 715, thus accelerating a flow speed of the aromatic liquid in the shrinkage conduit 731 of the nozzle 73. Thereafter, a flow of the gas sprays out of the outlet 755 of the receiving groove 751 via the narrow conduit 733 and flows through the suction mouth 753 at a highest flow speed so as to produce a pressure difference of the aromatic liquid around the suction mouth 753, thus providing a suction force to the sucking tube 90. Then, the aromatic liquid is drawn into the receiving groove 751 from the bottle 9 via the suction mouth 753 and is sprayed out of the outlet 755 from the receiving groove 751 by the gas from the nozzle 73, thus diffusing the scents by using Venturi effect. For example, the gas is successively delivered and sprayed out the narrow conduit 733 at the highest flow speed via a small-diameter portion of the shrinkage conduit 731 so that a flow speed of the gas increases because of a change (such as from the shrinkage conduit 731 to the narrow conduit 733) of a cross-sectional area of the nozzle 73 and a pressure decreases. The gas sprays out of the outlet 755 through the suction mouth 753 so as to produce a pressure drop (a pressure difference) around the suction mouth 753, thus drawing the aromatic liquid to the suction mouth 753 via the sucking tube 90. Thereafter, the aromatic liquid flows into the slit 757 of the nozzle 73 and is atomized and sprayed to the first orifice 711 and the spray hose 79 by a high-speed gas of the receiving groove 751 and the nozzle 73 from the outlet 755, thus diffusing the scents.

Accordingly, the scent diffuser of the present invention diffuses the scents by pumping the gas to the diffusing device 7. Preferably, the bottle 9 is removable from the connection portion 771 of the holder 77, thus replacing the bottle 9 quickly.

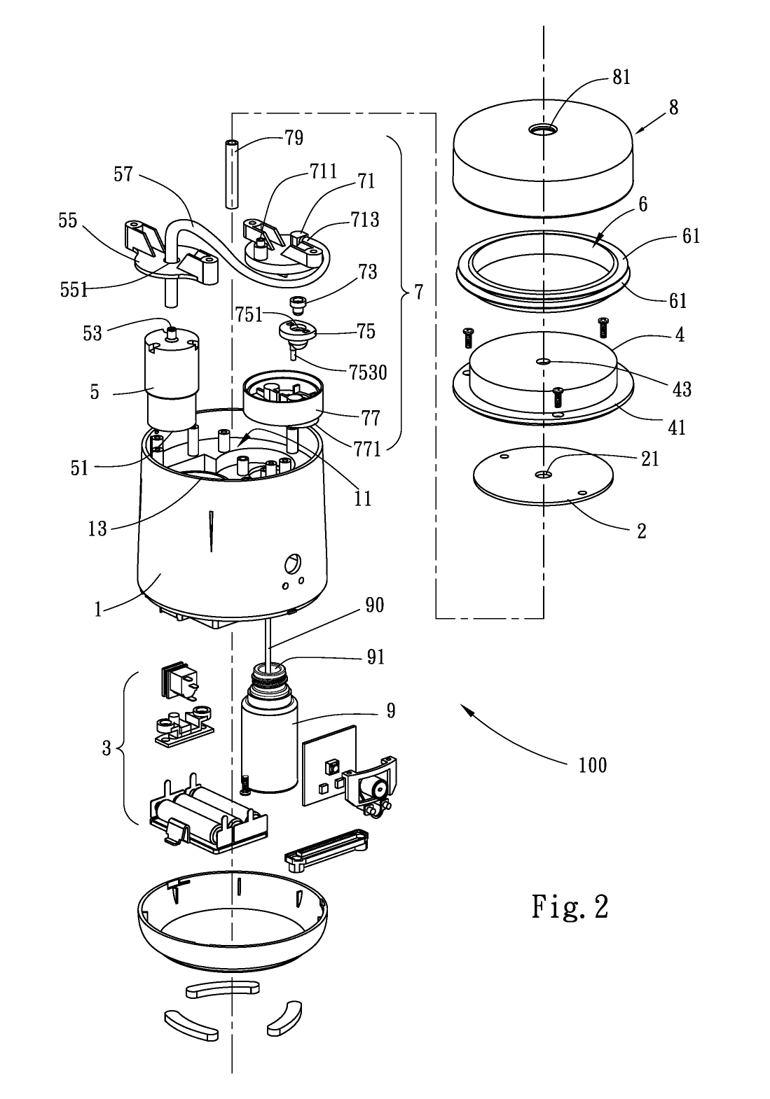

Referring to FIGS. 2-8, the bottle 9 is connected with the diffusing device 7 by way of the sucking tube 90, the bottle 9 includes the sucking tube 90 and the second opening 91 configured to removably connect with the connection portion 771 of the holder 77. The bottle 9 is configured to accommodate the aromatic liquid, and the sucking tube 90 is housed in the bottle 9 and its free end is coupled with the suction mouth 753.

The aromatic liquid is any one of essential oil, fragrance oil, essence, flower essence, perfume, and fragrance agent so as to produce the scents quickly after being atomized.

With reference to FIGS. 2-8, the cap 71 has a hook 717 extending downward from the bottom of the cap 71 adjacent to the third orifice 715, and the spraying member 75 has a recess 757 formed on an end thereof. When the cap 71 is connected with the holder 77, the hook 717 is retained with the recess 757 of the spraying member 75, and the third orifice 715 of the cap 71 communicates with the upper end of the nozzle 73 so that the gas flows through the shrinkage conduit 731 of the nozzle 73 from the third orifice 715.

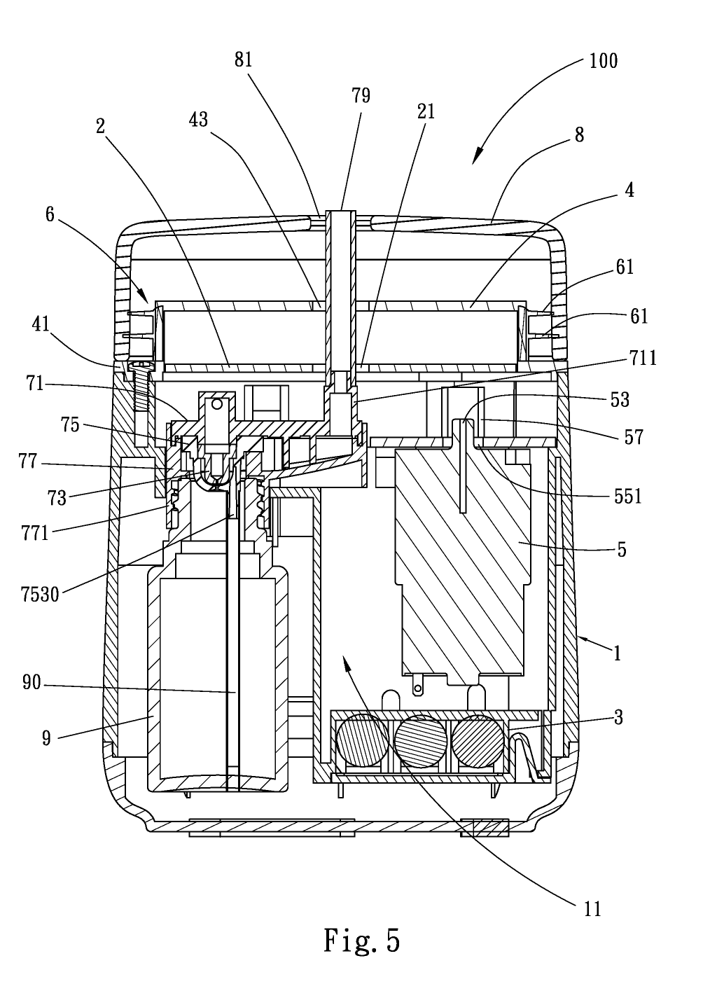

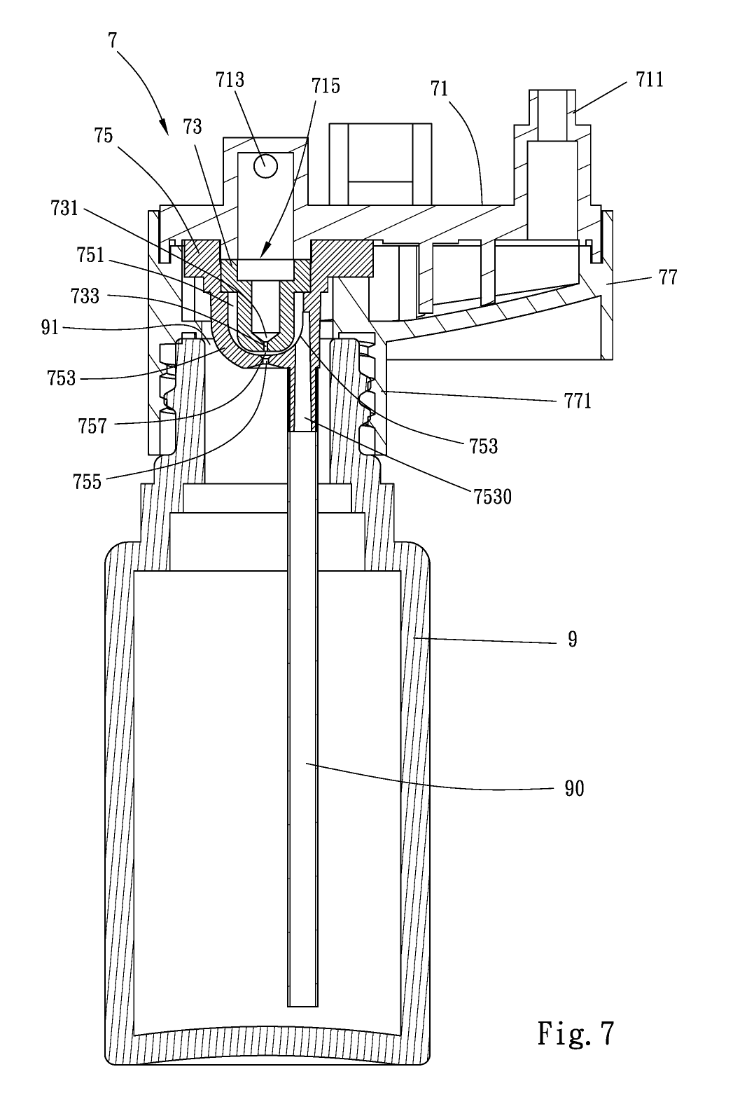

As shown in FIGS. 5 and 7, the receiving groove 751 of the spraying member 75 is in a U shape so that the slit 757 is defined between the nozzle 73 and the spraying member 75, the aromatic liquid flows into the slit 757 from the sucking tube 90 via the suction mouth 753, and the aromatic liquid is atomized and sprayed out of the outlet 755 by using the gas of the narrow conduit 733.

With reference to FIGS. 2, 4, and 6, the spraying member 75 has a drawing tube 7530 extending from the suction mouth 753 and communicating with the sucking tube 90, wherein the suction mouth 753 is in communication with the sucking tube 90 via the drawing tube 7530.

Referring to FIGS. 2, 5, and 8, the scent diffuser 100 further comprises a covering member 55 and a connecting tube 57, wherein the covering member 55 has an aperture 551 so that when the covering member 55 is coupled on a top of the pump 5, the connecting tube 57 inserts through the aperture 551, wherein a first end of the connecting tube 57 communicates with the outflow segment 53 of the pump 5, and a second end of the connecting tube 57 communicates with the second orifice 713 of the cap 71.

As shown in FIGS. 2, 3 and 5, the scent diffuser 100 further comprises an illumination device 2 and a first case 4. The illumination device 2 includes a light emitting element (not shown) and a circuit board. The illumination device 2 is electrically connected with the power connection unit 3 so as to emit lights, and the illumination device 2 is housed in the accommodation chamber 11 of the body 1. The first case 4 is made of transparent material or translucent material. The first case 4 includes a light guide loop 41 extending around an outer peripheral side thereof, such that when the first case 4 is connected with the body 1, the power connection unit 3 is coupled with the power supply so as to supply the power and to emit the lights. The light emitting element (not shown) of the illumination device 2 emits the lights, the first case 4 receives the lights from the light emitting element and the light guide loop 41 guides and sends the lights outside the scent diffuser 100, hence the scent diffuser 100 has decoration effect by way of the light guide loop 41. The light emitting element (not shown) of the illumination device 2 is light-emitting diode (LED) or various light bulbs.

As illustrated in FIGS. 2, 3, and 5, the scent diffuser 100 further comprises a support loop 6 and a second case 8 which is hollow. The support loop 6 includes multiple abutting ribs 61 separately surrounding an outer wall thereof. When the support loop 6 is fitted with an outer peripheral side of the first case 4 and the second case 8 covers the first case 4, an inner wall of the second case 8 removably retains with the multiple abutting ribs 61 of the support loop 6, and a bottom of the second case 8 retains on a top of the light guide loop 41, such that the upper end of the body 1 is located on a bottom of the light guide loop 41 so that the outer peripheral side of the first case 4 exposes the scent diffuser 100. Preferably, the second case 8 removes from or covers on the support loop 6 easily and is connected with the body 1 temporarily. The support loop 6 is made of flexible material or deformable material, and the flexible material or the deformable material is any one of silicone rubber, rubber, and plastic.

With reference to FIGS. 2 and 5, the illumination device 2 further includes a first hole 21, the first case 4 includes a second hole 43, and the second case 8 includes a spout 81, wherein the spray hose 79 inserts through the first hole 21, the second hole 43 and the spout 81 so as to communicate with the external environment outside the scent diffuser 100, and the spout 81 guides aromatic atomized gas to flow toward the external environment.

Referring to FIGS. 2-3, 5, and 7, the connection portion 771 is female threads, and the bottle 9 includes male threads arranged on an outer wall thereof so as to screw with or unscrew from the connection portion 771.

Thereby, the scent diffuser 100 diffuses the scents quickly after spraying and atomizing the aromatic liquid by using the diffusing device.

While the preferred embodiments of the invention have been set forth for the purpose of disclosure, modifications of the disclosed embodiments of the invention as well as other embodiments thereof may occur to those skilled in the art. The scope of the claims should not be limited by the preferred embodiments set forth in the examples, but should be given the broadest interpretation consistent with the description as a whole.

What is claimed is:

1. A scent diffuser comprising:
a body including an accommodation chamber defined in the body and a first opening formed on a top of the accommodation chamber;
a power connection unit housed in the accommodation chamber of the body;
a pump including an air inflow segment and an air outflow segment, and the pump being housed in the accommodation chamber of the body and electrically connected with the power connection unit;
a diffusing device accommodated in an upper end of the body, and the diffusing device including a cap, a nozzle, a spraying member, a holder, and a spray hose;
the cap having a first orifice, a second orifice, and a third orifice, the second orifice being formed on a top of the cap and communicating with the outflow segment of the pump, and the third orifice being formed on a bottom of the cap and communicating with the second orifice;
the spraying member having a receiving groove, a suction mouth, and an outlet, wherein the receiving groove is defined in the spraying member, the outlet communicates with a first side of the receiving groove, and the suction mouth is in communication with a second side of the receiving groove;
wherein the nozzle has a shrinkage conduit and a narrow conduit, the shrinkage conduit communicates with the narrow conduit, the spraying member is connected in the holder, the nozzle is accommodated in the receiving groove of the spraying member, a slit is defined between the nozzle and the receiving groove, and a free end of the narrow conduit is proximate to the outlet;
wherein the holder has a connection portion arranged on a side of the holder; and
wherein the cap is coupled on a top of the holder, the third orifice of the cap communicates with an upper end of the nozzle, and the first orifice of the cap communicates with an interior of the diffusing device and an exterior outside the diffusing device; a first side of the spray hose communicates with the first orifice, and a second side of the spray hose is in communication with an external environment outside the scent diffuser.

2. The scent diffuser as claimed in claim 1, wherein a bottle is connected with the diffusing device by way of a sucking tube, the bottle includes the sucking tube and a second opening configured to removably connect with the connection portion of the holder; the bottle is configured to accommodate aromatic liquid, and the sucking tube is housed in the bottle and a free end of the sucking tube is coupled with the suction mouth.

3. The scent diffuser as claimed in claim 1, wherein the cap has a hook extending downward from the bottom of the cap adjacent to the third orifice, and the spraying member has a recess formed on an end thereof; when the cap is connected with the holder, the hook is retained with the recess of the spraying member, and the third orifice of the cap communicates with the upper end of the nozzle.

4. The scent diffuser as claimed in claim 1, wherein the receiving groove of the spraying member is in a U shape so that the slit is defined between the nozzle and the spraying member.

5. The scent diffuser as claimed in claim 1, wherein the spraying member has a drawing tube extending from the suction mouth and communicating with the sucking tube.

6. The scent diffuser as claimed in claim 1 further comprising a covering member and a connecting tube, wherein the covering member has an aperture so that when the covering member is coupled on a top of the pump, the connecting tube inserts through the aperture, wherein a first end of the connecting tube communicates with the outflow segment of the pump, and a second end of the connecting tube communicates with the second orifice of the cap.

7. The scent diffuser as claimed in claim 1 further comprising an illumination device and a first case, wherein the illumination device includes a light emitting element, the illumination device is electrically connected with the power connection unit so as to emit lights, and the illumination device is housed in the accommodation chamber of the body; the first case is made of transparent material or translucent material, and the first case includes a light guide loop extending around an outer peripheral side thereof.

8. The scent diffuser as claimed in claim 7 further comprising a support loop and a second case which is hollow; the support loop includes multiple abutting ribs separately surrounding an outer wall thereof; when the support loop is fitted with an outer peripheral side of the first case and the second case covers the first case, an inner wall of the second case removably retains with the multiple abutting ribs of the support loop, and a bottom of the second case retains on a top of the light guide loop, such that the upper end of the body is located on a bottom of the light guide loop so that the outer peripheral side of the first case exposes the scent diffuser.

9. The scent diffuser as claimed in claim 8, wherein the illumination device further includes a first hole, the first case includes a second hole, and the second case includes a spout, wherein the spray hose inserts through the first hole, the second hole and the spout so as to communicate with the external environment outside the scent diffuser.

10. The scent diffuser as claimed in claim 1, wherein the connection portion is female threads, and the bottle includes male threads arranged on an outer wall thereof so as to screw with or unscrew from the connection portion.
